(19) United States
(12) Patent Application Publication
(10) Pub. No.: US 2007/0134220 A1
(43) Pub. Date: Jun. 14, 2007

(54) Lactobacillus fermentum strain and uses thereof

(75) Inventors: Alain Servin, Nanterre (FR); Gilles Chauviere, Antony (FR); Marie-Helene Polter, Ivry Sur Seine (FR); Vanessa Le Moal, Bievres (FR); Bruno Gastebois, Chartres (FR)

Correspondence Address: LERNER, DAVID, LITTENBERG,;KRUMHOLZ & MENTLIK, 600 SOUTH AVENUE WEST, WESTFIELD, NJ, 07090, US

(73) Assignees: Axcan Pharma S.A., Houdan (FR); Institut National de la Sante et de la Recherche Medicale (INSERM), Paris (FR)

(21) Appl. No.: 10/551,566

(22) PCT Filed: Apr. 2, 2003
(86) PCT No.: PCT/IB03/01739

§ 371 Date: Jul. 10, 2006

(51) Int. Cl. A61K 35/74; C12N 1/20

(52) U.S. Cl. 424/93.45; 435/252.9

(57) ABSTRACT

The present invention concerns a new strain of Lactobacillus fermentum (LB-f) that is useful in preventing or treating gastrointestinal disorders in mammals, especially in humans. Also, this strain can be used as a dietary product that is beneficial to the wellbeing and health of mammals, including humans.

The present invention relates to a new strain of the Lactobacillus genus, and uses thereof for medical or dietary purposes.

In particular, the invention concerns a new strain of Lactobacillus fermentum (LB-f) that is useful in preventing or treating gastrointestinal disorders in mammals, especially in humans.

Also, this strain can be used as a dietary product that is beneficial to the wellbeing and health of mammals, including humans.

Microorganisms, and more particularly bacteria, that produce lactic acid as a major metabolic compound have been known for a long time. These bacteria may be found in milk, in milk processing factories, in living or decaying plants, as well as in the intestine of mammals, especially humans. These microorganisms, brought together under the generic formula <<lactic acid bacteria>>, represent a rather inhomogeneous group and comprise e.g. the genera Lactococcus, Lactobacillus, Streptococcus, Bifidobacterium, Pediococcus, etc. . . .

Lactic acid bacteria have been utilized as fermenting agents for the preservation of food, taking benefit of a low pH and the action of fermentation products generated during the fermentative activity thereof to inhibit the growth of spoilage bacteria. In this context, lactic acid bacteria have been used for preparing a variety of different foodstuff such as cheeses, yogurts and other fermented dairy products from milk.

Quite recently, lactic acid bacteria have attracted a great deal of attention in that some strains have been found to exhibit valuable properties to mammals, including humans, upon ingestion. In particular, specific strains of the genus Lactobacillus or Bifidobacterium have been found to be able to colonize the intestinal mucosa and to assist in the maintenance of the wellbeing of mammals.

For instance, European patent application EP 0 203 586, in the name of Pioneer Hi-Bred International, discloses a composition for treating gastrointestinal diseases, e.g., caused by enterotoxigenic strains of Escherichia coli, in animals, said composition containing L. fermentum ATCC 53113 or mutants thereof. Animals as referred to therein are domestic animals, such as pigs, cows, sheep, goats, and horses. L. fermentum ATCC 53113 was isolated from the gut of a healthy newborn pig.

During the last few years, research has also focused on the potential use of lactic acid bacteria as probiotic agents.

Probiotics are considered to be viable microbial preparations which promote the health of mammals, especially humans. Probiotics are deemed to attach to the intestine's mucosa, colonize the intestinal tract and likewise prevent attachment of harmful microorganisms thereon. A crucial prerequisite for their action resides in that they have to reach the gut's mucosa in a proper and viable form and do not get destroyed in the upper part of the gastrointestinal tract, especially by the influence of the low pH prevailing in the stomach.

In particular, European patent application EP 1 034 787, in the name of Société des Produits Nestlé S.A., discloses new strains belonging to the Lactobacillus genus, that are useful for preventing diarrhoea, by inhibiting intestine colonization by pathogenic bacteria. These strains, especially L. paracasei CNCM 1-2116, can be used for preparing pharmaceutical or dietary compositions.

Furthermore, International patent application WO 02/45727, in the name of Plbio Co., Ltd., describes lactic acid bacteria capable of inhibiting activities and growth of Helicobacter pylori causing stomach ulcer and adhesion to the gastric mucosa. The lactic acid bacteria disclosed therein as being able to suppress stomach ulcers, are selected from the group of L. coprophilus, Enterococcus durans, Streptococcus faecalis, and L. fermentum. According to this disclosure, the lactic acid bacteria can be used in pharmaceutical compositions, in cosmetic preparations, for instance for treating acne, as well as in food additives, that can be added to, e.g., yogurts, dairy goods, cheeses, and the like.

In view of their potential properties, strains of lactic acid bacteria appear to be very valuable both for medical and dietary purposes.

In this context, a strain belonging to the Lactobacillus genus, namely L. acidophilus LB, and exhibiting interesting anti-diarrhoea properties, is currently used in France in the commercially-available “Lactéol®” pharmaceutical compositions. Nevertheless, this LB strain has a number of drawbacks related to the very specific conditions required to culture. Among these conditions, the composition of the culture medium should be mentioned, this medium having to be enriched by compounds from bovine origin such as lactosemum, bovine peptone, and casein. Moreover, before cultivating the LB strain, it is required to implement preliminary steps for appropriately preparing the culture medium by sterilization and filtration thereof, so that a clarified culture medium is obtained. Also, cultivating the LB strain is a relatively long and costly process, with high energetic demands; it produces a lot of organic waste that has to be collected and treated.

Therefore, there is a desire in the art for providing new strains of lactic acid bacteria that are at least as beneficial to the wellbeing and health of mammals, especially humans, as is the LB strain, but that have advantageous features compared thereto, especially in terms of conditions of culture.

To this end, the present invention provides a novel microorganism, namely a lactic acid bacterial strain belonging to the genus Lactobacillus, having the capability of preventing colonization of the stomach and the intestine by pathogenic bacteria, responsible for gastrointestinal disorders, and being easy to cultivate for industrial purposes.

The Lactobacillus strain of the invention is thus valuable for the wellbeing and health of mammals, including animals and humans, preferably humans, and more preferably infants.

In this respect, said Lactobacillus strain is a biotherapeutic agent, i.e., a biological agent which exhibits a therapeutic activity of interest.

Moreover, the Lactobacillus strain of the present invention is of high interest as far as safety standards are concerned, especially with regard to potential contaminating viral agents or unconventional transmissible pathogens. In this connection, said strain can be consumed or administered, as described hereunder, by mammals, more particularly by humans, without any risk.

In a first aspect, the present invention relates to a Lactobacillus fermentum strain (LB-f strain), deposited at the CNCM (Paris, France) on Mar. 27, 2003, under registration number I-2998.

This LB-f strain is herein described by phenotypic and genotypic features.

On the one hand, the LB-f strain exhibits at least the following phenotypic characters:
regular, non sporing, Gram-positive rod;
heterofermenting;
catalase negative;
L (+)-lactic acid-producing.

On the other hand, the LB-f strain is genotypically characterized by a sequence of 16S ribosomal DNA (rDNA), which comprises a nucleotide sequence selected from:
SEQ ID No. 1;
its complementary sequence; and
sequences identical at least at 98.1% to SEQ ID No. 1 or to its complementary sequence.

As used herein the term “complementary” means that, for example, each base of a first nucleotide sequence is paired with the complementary base of a second nucleotide sequence whose orientation is reversed. The complementary bases are A and T (or A and U) or C and G.

The terms <<nucleic acids>> and <<nucleotide sequences>> are used interchangeably according to their conventional meaning in the technical field of the invention.

According to a first embodiment, nucleotide sequences also encompassed by the present invention are identical at least at 98.5%, and preferably at least at 99% to SEQ ID No. 1 or to its complementary sequence.

According to a second embodiment, these nucleotide sequences are identical at least at 99.5%, and preferably at least at 99.8% to SEQ ID No. 1 or to its complementary sequence.

By “sequence identity”, it is herein referred to the identity between two nucleic acids.

Sequence identity can be determined by comparing a position in each of the two nucleotide sequences which may be aligned for the purposes of comparison. When a position in the compared sequences is occupied by the same base, then the sequences are identical at that position. A degree of sequence identity between nucleic acid sequences is a function of the number of identical nucleotides at positions shared by these sequences. Since two nucleotide sequences may each (i) comprise a sequence (i.e., a portion of a complete nucleotide sequence) that is similar, and (ii) may further comprise a sequence that is divergent, sequence identity comparisons between two or more nucleotide sequences over a “comparison window” refers to the conceptual segment of at least 20 contiguous nucleotide positions wherein a nucleotide sequence may be compared to a reference nucleotide sequence of at least 20 contiguous nucleotides and wherein the portion of the nucleotide sequence in the comparison window may comprise additions or deletions (i.e., gaps) of 20 percent or less compared to the reference sequence (which does not comprise additions or deletions) for optimal alignment of the two sequences.

To determine the percent identity of two nucleic acid sequences, the sequences are aligned for optimal comparison. For example, gaps can be introduced in the sequence of a first nucleic acid sequence for optimal alignment with the second nucleic acid sequence. The nucleotides at corresponding nucleotide positions are then compared. When a position in the first sequence is occupied by the same nucleotide as the corresponding position in the second sequence, the nucleic acids are identical at that position.

The percent identity between the two sequences is a function of the number of identical positions shared by the sequences.

Hence % identity=[number of identical positions/total number of overlapping positions]×100. The percentage of sequence identity is thus calculated according to this formula, by comparing two optimally aligned sequences over the window of comparison, determining the number of positions at which the identical nucleic acid base (e.g., A, T, C, G, U, or I) occurs in both sequences to yield the number of matched positions (the “number of identical positions” in the formula above), dividing the number of matched positions by the total number of positions in the window of comparison (e.g., the window size) (the “total number of overlapping positions” in the formula above), and multiplying the result by 100 to yield the percentage of sequence identity.

In this comparison, the sequences can be the same length or may be different in length. Optimal alignment of sequences for determining a comparison window may be conducted by the local homology algorithm of Smith and Waterman (1981), by the homology alignment algorithm of Needleman and Wunsh (1972), by the search for similarity via the method of Pearson and Lipman (1988), by computerized implementations of these algorithms (GAP, BESTFIT, FASTA and TFASTA in the Wisconsin Genetics Software Package Release 7.0, Genetic Computer Group, 575, Science Drive, Madison, Wis.), or by inspection.

The best alignment (i.e., resulting in the highest percentage of identity over the comparison window) generated by the various methods is selected.

The percentage of sequence identity of a nucleic acid sequence to a nucleotide sequence of reference can also be calculated using BLAST software (Version 2.06 of September 1998) with the default or user defined parameter.

In a second aspect, the present invention concerns a method for cultivating a LB-f strain as defined above, comprising at least:
a) providing a culture medium containing at least lactose and yeast extract;
b) cultivating said LB-f strain in said culture medium under fermenting conditions; and
c) recovering the thus obtained culture of the LB-f strain.

All the steps of such a method are conventional in the technical field of the invention, and can be performed by a skilled artisan in the light of his general knowledge.

Interestingly, a suitable culture medium is free of compounds from bovine origin. Such a medium free of derivatives from bovine origin ensures enhanced safety of the thus obtained culture of LB-f strain.

Advantageously, the method for cultivating the LB-f strain is simple, easy to perform, and does not require sterilization and filtration of the medium prior to culture. Moreover, waste products are considerably reduced.

According to a first embodiment, said culture medium contains lactose at a concentration range of about 50 to about 100 g/l.

According to a second embodiment, said culture medium contains yeast extract at a concentration range of about 5 to about 20 g/l.

According to a third embodiment, said fermenting conditions in step b) are pH-regulated, said pH ranging between about 4.5 and 5.5.

Also encompassed by the second aspect of the invention is a method as described above, further comprising separating the biomass from the culture supernatant (LB-f-SCS) by centrifugating said culture of LB-f strain recovered in step c).

Yet encompassed by the second aspect of the invention is a method as described above, wherein, once centrifugation has been performed, said biomass and/or said LB-f-SCS are recovered.

In a third aspect, the present invention is directed to a Lactobacillus fermentum culture supernatant (LB-f-SCS) obtainable by the aforementioned method for cultivating a LB-f strain.

In a fourth aspect, the invention is related to a LB-f strain or a LB-f-SCS as defined above, for use as a medicine.

In particular, said medicine is used for preventing and/or treating gastrointestinal disorders.

Of great significance is the fact that the medicine according to the invention does not necessitate the supplemental use of antibiotics and relies rather upon “natural” mechanisms of controlling the pathogens, in particular by preventing same from binding to gut-associated tissue.

This is significant in the face of growing public concern over the misuse of antibiotics, and the effect that over-intake of antibiotics is given to have on the population's health.

The gastrointestinal disorders against which the present invention is effective can be any in which the underlying etiology is microbial, for example, bacterial or viral in nature.

Alternatively, the present invention is also useful in mammals, including humans, where the normal gut flora has been eliminated or unbalanced, for example, following severe viral gastroenteritis or high dose antibiotic therapy, in order to aid in the restoration of the normal gut flora and prevent colonization by opportunistic pathogens.

Preferably, in the context of the invention, the expression “gastrointestinal disorders” refers to disorders or diseases that are selected from ulcers and infections due to Helicobacter pylori, intestinal inflammatory diseases, such as ulcerous colitis, Crohn's disease and pouchitis, irritable bowel syndrome, steatohepatitis, hepatic steatosis, and infectious diarrhoea.

In a fifth aspect, the invention concerns the use of a LB-f strain or a LB-f-SCS as described above, for the manufacture of a medicine for preventing and/or treating gastrointestinal disorders.

In a sixth aspect, the present invention relates to the use of a LB-f strain or a LB-f-SCS as disclosed herein, as a dietary product.

In a seventh aspect, the invention is directed to a pharmaceutical composition comprising a LB-f strain or a LB-f-SCS, and a pharmaceutically acceptable carrier.

The composition of the present invention is highly desirable in that the LB-f strain of the invention is non-pathogenic and should thereby render unlikely the occurrence of any deleterious effects due thereto.

According to a first embodiment, said LB-f strain is present in the pharmaceutical composition an amount from about 10⁹ to about 10¹² bacteria/g, preferably from about 10⁹ to about 10¹¹ bacteria/g, and more preferably from about 10⁹ to about 10¹⁰ bacteria/g.

According to a second embodiment, said LB-f-SCS is present in the pharmaceutical composition in an amount of at least about 100 mg per gram of composition.

According to a third embodiment, the pharmaceutical composition of the invention is ingestible.

Such an ingestible composition is preferably in a form selected from tablets, liquid bacterial suspensions, dried oral supplements, wet oral supplements, dry tube feeding, wet tube feeding.

According to an eighth aspect, the present invention is related to a method for treating or preventing gastrointestinal disorders in mammals, especially humans, that are in need of such treatment.

This method comprises administering to a mammal in need of such treatment a pharmaceutically effective amount of a medicine selected from the group of:

a LB-f strain as defined above; or

a LB-f-SCS as described herein; or

a pharmaceutical composition as previously mentioned.

As used herein, the expression “in need of such treatment” refers to a mammal, including a human, having, or being at risk of having, gastrointestinal disorders.

According to a first embodiment, administration of said medicine is performed orally.

To do so, a suitable medicine is in a form selected from tablets, liquid bacterial suspensions, dried oral supplements, wet oral supplements, dry tube feeding, wet tube feeding.

In a ninth aspect, the present invention concerns a dietary composition comprising a LB-f strain or a LB-f-SCS as defined above, and a food carrier, such as milk, cheese, yogurts, and the like.

According to a first embodiment, said LB-f strain is present in the dietary composition in an amount from about 10⁵ to about 10⁹ bacteria/g, preferably from about 10⁶ to about 10⁸ bacteria/g, and more preferably from about 10⁶ to about 10⁷ bacteria/g.

According to a second embodiment, said LB-f-SCS is present in the dietary composition in an amount of less than about 100 mg per gram of composition.

A dietary composition according to the present invention is advantageously ingestible.

In this respect, said ingestible composition is preferably selected from milk, yogurt, curd, cheese, fermented milks, fermented milk-based products, ice-creams, fermented cereal-based product, milk-based powders, infant formulae.

Generally, compositions of the present invention, either pharmaceutical or dietary, may be in a liquid, solid, lyophilized, or gel form.

On the one hand, in solid oral dosage forms, the compositions may comprise the LB-f strain or the LB-f-SCS, together with an appropriate carrier, that is either a pharmaceutically acceptable carrier or a food carrier. Such a carrier may be in a form chosen among: aqueous or non-aqueous liquids, and solids.

Also, the solid compositions may contain inert diluents such as sucrose, lactose, starch, or vermiculite, as well as lubricating agents. Lubricating agents help the compositions to pass through the gut.

In the case of capsules, tablets and pills, the unit dosage forms may also comprise buffering agents.

Others forms of oral administration may also be prepared with a gastric or an enteric coating which would prevent dissolution of the compositions until reaching the stomach or the intestines, respectively.

On the other hand, liquid dosage forms for oral administration may comprise an enterically-coated capsule containing the liquid dosage form.

Suitable liquid forms include emulsions, suspensions, solutions, syrups, and elixirs containing inert diluents commonly used in the art, such as purified water, sugars, polysaccharides, silicate gels, gelatin, or an alcohol. Inert diluents do not actively participate in the therapeutic or dietary effect of interest. Besides the inert diluents, such compositions can also include adjuvants, for instance wetting, emulsifying, suspending, sweetening, flavouring, and perfuming agents.

Those of ordinary skill in the art will know of other suitable diluents and dosage forms, or will be able to ascertain such, using routine experimentation.

Further, the administration or ingestion of the compositions of the invention can be carried out using standard techniques common to those of ordinary skill in the art.

The present invention is illustrated, without being limited, by the following drawings.

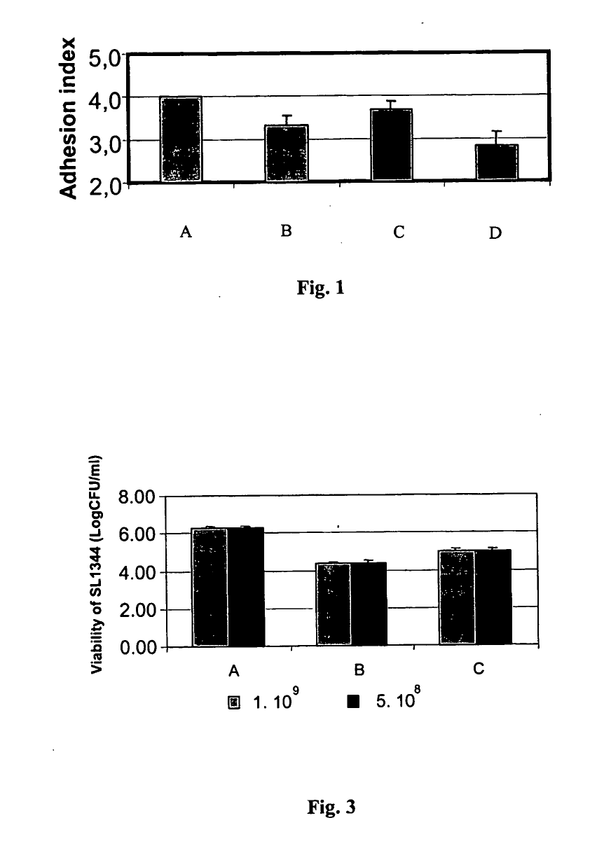

FIG. 1: Graphic representation of Lactobacillus fermentum LB-f adhesion to Caco-2/TC7 cells.

A: living LB-f (1.10⁹ bacteria/well)

B: living LB-f (2.10⁸ bacteria/well)

C: killed LB-f (2.10⁹ bacteria/well)

D: killed LB-f (1.10⁹ bacteria/well)

FIG. 2: Graphic representation of inhibition of cell-association of strain DAEC C1845, compared to controls of cell-association performed in:

A: DMEM

B: MRS-HCl pH 4.5

1: living LB

2: living LB-f

3: killed LB

4: killed LB-f

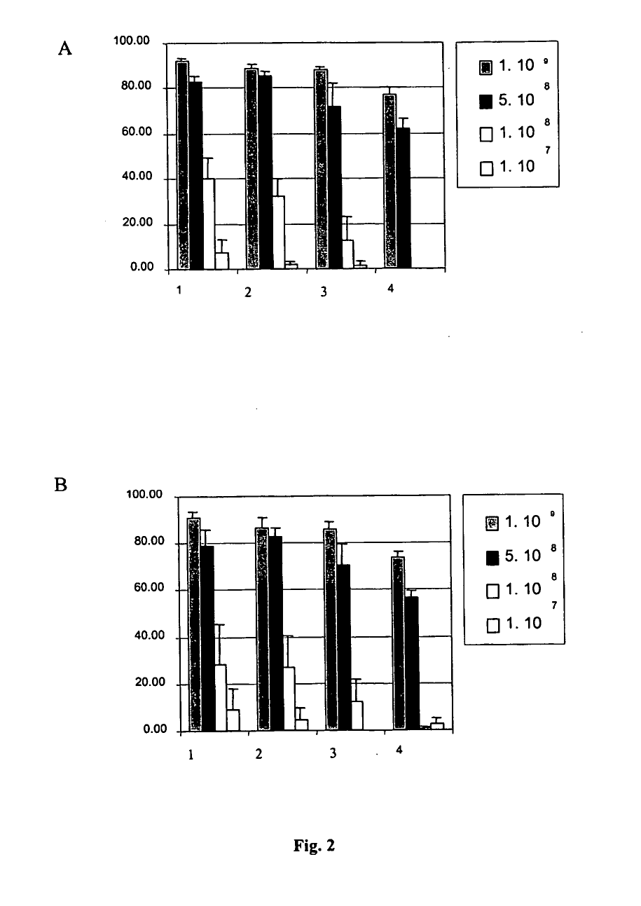

FIG. 3: Graphic representation of invasion of Salmonella serovar Typhimurium in the presence of Lactobacillus (bacteria and spent culture).

A: SL1344 control

B: living LB-f

C: killed LB-f

FIG. 4: Graphic representation of viability, adhesion and invasion of SL1344 after contact with culture supernatants during 1 hour.

A: DMEM control

B: MRS control

C: LB-SCS unheated

D: LB-f-SCS unheated

E: LB-SCS heated at 100° C.

F: LB-f-SCS heated at 100° C.

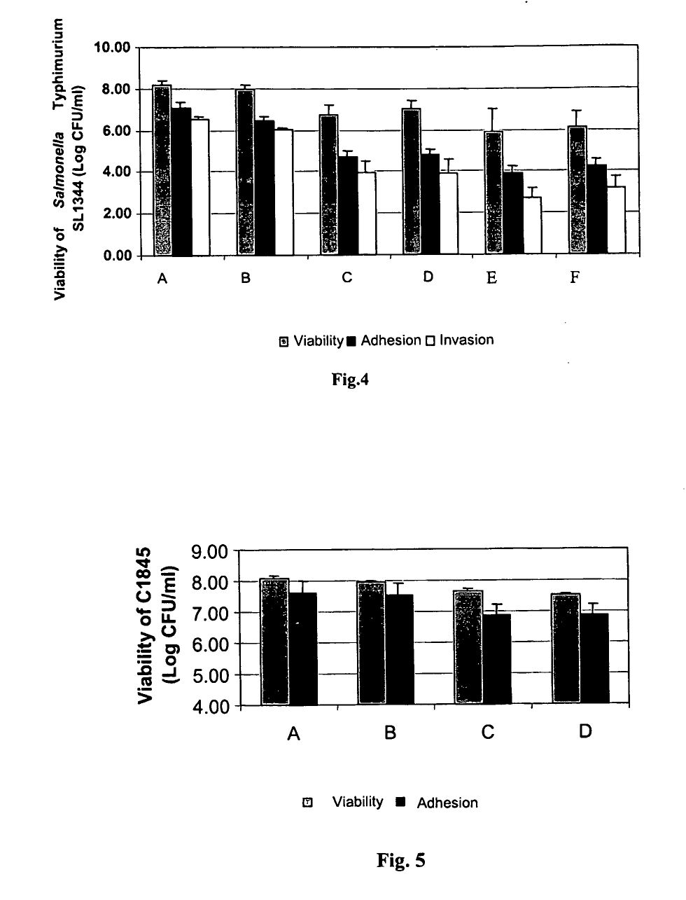

FIG. 5: Graphic representation of inhibition of viability and adhesion of DAEC C1845, after contact during 1 hr with culture supernatants LB-f-SCS, compared to DMEM and MRS controls.

A: DMEM control

B: MRS control

C: LB-f-SCS unheated

D: LB-f-SCS heated at 100° C.

FIG. 6: Graphic representation of H. pylori viability after contact during 2 hrs with LB-f-SCS, compared to BHI and MRS controls.

A: BHI control

B: MRS control

C: LB-f-SCS unheated

D: LB-f-SCS heated at 100° C.

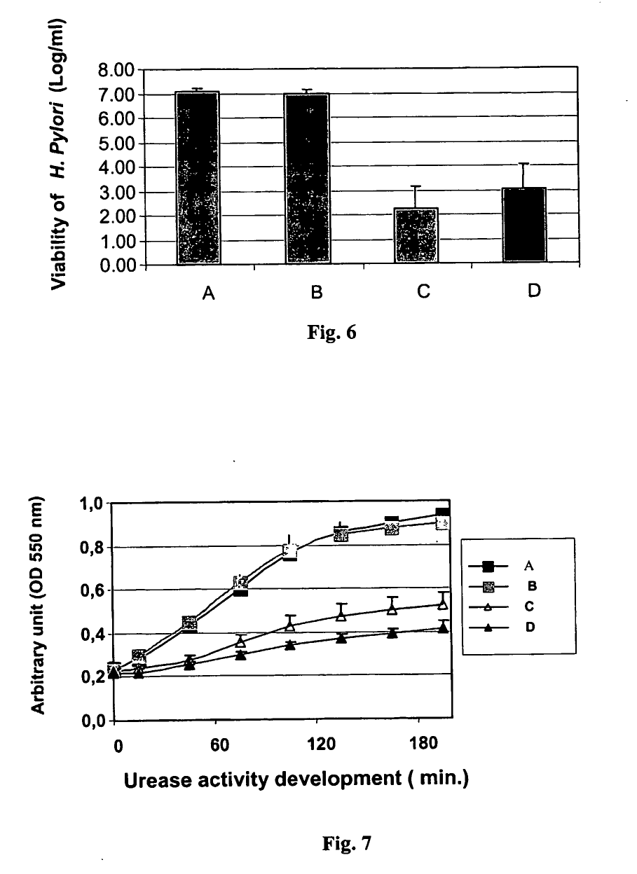

FIG. 7: Graphic representation of development of urease activity after contact during 2 hrs with H. pylori.

A: BHI control

B: MRS control

C: LB-f-SCS unheated

D: LB-f-SCS heated at 100° C.

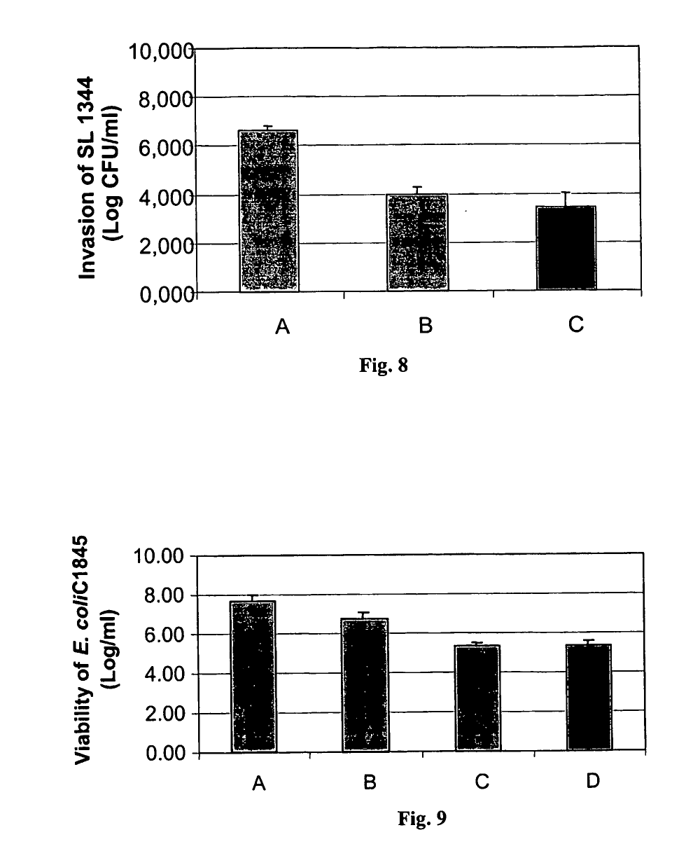

FIG. 8: Graphic representation of cell-invasion of Salmonella SL1344 after treatment for 1 hr with LB-f-SCS, said treatment being performed after infection, compared to DMEM control.

A: DMEM control

B: LB-f-SCS unheated

C: LB-f-SCS heated ay 100° C.

FIG. 9: Graphic representation of cell-adhesion of DAEC C1845 after treatment for 1 hr with LB-f-SCS, said treatment being performed after infection, compared to DMEM and MRS controls.

A: DMEM control

B: MRS control

C: LB-f-SCS unheated

D: LB-f-SCS heated at 100° C.

The above disclosure generally describes the present invention.

A more complete understanding can be obtained by reference to the following experimental procedures and results which are provided herein for purposes of illustration only and are not intended to be limiting unless otherwise specified.

I. Experimental Procedures

I.1 Pathogens:

Salmonella typhimurium SL 1344 (Finlay and Falkow, 1997) was a gift of B. A. D. Stocker (Stanford, Calif.), Escherichia coli C1845 was a gift of S. Bilge (University of Washington, Seattle); enterotoxigenic E. coli (ETEC) strains H10407 expressing Coli Factor Adhesin type 1 (CFA/I) was provided by A. Darfeuille-Michaud (Faculté Médecine-Pharmacie, Clermont-Ferrand).

E. coli strains were grown on CFA-agar containing 1% Casamino Acids (Difco Laboratories, Detroit, Mich.), 0.15% yeast extract, 0.005% magnesium sulfate, and 0.0005% manganese chloride in 2% agar for 18 hours at 37° C.

For radiolabeling, bacteria were subcultured twice at 37° C. for 24 hours in CFA or Trypticase Soya Agar (TSA) broth. They were metabolically labeled by the addition of ¹⁴C-acetic acid (Amersham, 94 mCi/mmol; 100 μCi per 10 ml tube).

S. typhimurium was cultured at 37° C. for 18 hours in Luria broth and an exponential culture of Salmonella was used for assays.

For radiolabeling assays, S. typhimurium was cultured 4 hours in Luria broth and then was subcultured at 37° C. for 45 min in methionine medium (Difco) with ³⁵S-methionine (Amersham 1000 Ci/mmol, 20 μCi/ml) for radiolabelling.

Strain of H. pylori was provided by I. Corthesy-Theulaz (Institute of Microbiology, Lausanne University, Lausanne, Switzerland). H. pylori strain 1101 was isolated from a patient suffering from functional dyspepsia and erosive gastritis (Corthézy-Theulaz et al., 1995).

H. pylori was grown on Brain-Heart Infusion (BHI)-agar plates containing 0.25% yeast extract (Difco Laboratories), 10% horse serum. Helicobacter culture was incubated upside down in a gas jar with microaerophilic atmosphere (Gas-generating kit, CampyGen, Oxoid Ltd) at 37° C. for 36 hours.

I.2 Cultured Cell Lines:

In the last few years, human intestinal cell lines have been established. These include HT-29 and Caco-2 cells established in 1964 and 1974, respectively, by Jorgen Fogh at the Memorial Sloan Kettering Cancer Center (New York, USA) (Fogh et al., 1977), which are both derived from human colonic adenocarcinomas. The clone Caco-2/TC7 was used here (Chantret et al., 1993). They display spontaneously in culture (Caco-2 and Caco-2/TC7) the following types of enterocyte differentiation specific to the human small intestine (Zweibaum et al., 1991):
cell polarisation
development of an apical brush border and tight junctions
   expression of intestinal hydrolases, e.g.: saccharase-isomaltase (SI), neutral aminopeptidase (NAp), dipeptidyl peptidase IV (DPP IV) . . . ,
production of structural proteins: villin . . . ,
presence of basolateral receptors: Vaso Active Intestinal Peptide (VIP), alpha2-adrenergic receptor . . . ,
presence of transport proteins: glucose transporter and transepithelial hydroelectrolytic transport.

Cells were routinely grown in Dulbecco modified Eagle's minimal essential medium (DMEM) (25 mM glucose) (InVitrogen, Cergy-Pontoise, France), supplemented with 15% heat-inactivated (30 min, 56° C.) fetal calf serum (InVitrogen, Cergy-Pontoise, France) and 1% non-essential amino acids (InVitrogen, Cergy-Pontoise, France). Cells were used at post-confluence after 15 days of culture (differentiated cells) for cell-association and cell-invasion assays using S. enterica serovar Typhimurium.

I.3 Lactobacilli Adhesion:

The required concentration of the bacterial suspension was made once they had been counted on a Petit Salumbéni cell. The cell layers were washed twice (2 ml) with sterile PBS. One millilitre of DMEM was placed on the cell layers and the same volume of the bacterial suspension was then added. They were left in contact for one hour with 10% CO₂ to preserve the integrity of the cell layer. The medium was removed at the end of incubation and the cell layers were washed five times (2 ml) with phosphate-buffered saline (PBS).

In each wash the well dishes were shaken round twenty times to remove non-adherent bacteria. Previous experiments have shown that further washes do not significantly reduce the adhesion ratio. The cell layers were then fixed in the following series of methanol baths: 70° C. 10 min, 95° C. 10 min and 100° C. 15 min. The cell layer were then dried, stained (Gram), dehydrated in xylene and mounted for microscopic observation (Eukitt). Each test was duplicated for three successive passages. The adherent bacteria were counted in 20 randomly chosen microscopic fields. The adherent bacteria were counted by two different technicians to prevent errors. Adhesion of Lactobacillus was graded on a 0 to 4 scale: 0, absence; ++, reduced number; +++, large number; ++++, high number.

LB-f strain was killed by incubating at 110° C. for 1 hour (see FIG. 1).

I.4 Association and Intracellular Inhibition of Enterovirulent Bacteria with Lactobacilli and Spent Culture Supernatant:

Inhibition Association was Conducted as Follows:

The Caco-2/TC7 monolayers were washed twice with PBS. ¹⁴C-radiolabelled bacteria were suspended in the culture medium.

For E. coli assays, incubations were conducted in presence of 1% D-mannose that inhibits type 1 pili adhesion. For evaluation of adhesion interference 250 μl of radiolabelled bacteria (4.108 CFU cells/ml), 250 μl of living or heat-killed L. fermentum LB-f with spent culture supernatant (10⁹ to 10⁵ CFU cells/ml as indicated) and 500 μl of DMEM were added to each well of the tissue culture plate. The plates were incubated at 37° C. in 10% CO₂/90% air for 3 h for E. coli and 1 h for Salmonella. The monolayers were then washed three times with sterile PBS. Associated bacteria and intestinal cells were dissolved in a 0.2 N NaOH solution. The level of bacterial association was evaluated by liquid scintillation counting. Each adherence assay was conducted in triplicate with three successive passages of Caco-2 cells by 2 technicians to prevent errors.

Inhibition of Salmonella Invasion was Performed as Follows:

Prior to infection, the Caco-2/TC7 were washed twice with PBS. A exponential culture of Salmonella was suspended in DMEM (1.10⁸ CFU/ml). 500 μl of this suspension and 500 μl of Lactobacillus (bacteria and spent culture) was added to each well of the tissue culture plate. The plates were incubated for 1 h at 37° C. in 10% CO₂/90% air and then washed three times with sterile PBS.

Internalization of enteroinvasive bacteria was determined by quantitative determination of bacteria located within the infected monolayers using the aminoglycoside antibiotic assay. After incubation, monolayers were washed twice with sterile PBS and, afterwards, incubated 60 min in a medium containing 100 μg/ml gentamicin. Bacteria that adhere to the Caco-2/TC7 brush border were rapidly killed, whereas those located within Caco-2 cells were not. The monolayer was washed with PBS and lysed with sterilized H₂O. Appropriate dilutions were plated on TSA to determine the number of viable cell-intracellular bacteria by bacterial colony counts.

Results are shown in FIG. 3.

I.5 Inhibition Assays of Bacterial Viability, Cell-Association and Cell-Invasion of Bacteria with Lactobacilli Supernatant:

The inhibition of cell-association or invasion of enteroinvasive bacteria by LB-SCS was determined by preincubating the pathogen (10⁸ CFU/ml) with control DMEM or concentrated LB-f-SCS for 1 hr at 37° C. After centrifugation (5,500×g, 10 min. at 4° C.), the bacteria were washed with PBS and resuspended in the PBS.

A 18 h old of Diffusely Adhesing E. Coli (DAEC), or exponential culture of Salmonella were centrifuged and suspended in DMEM. Colony count assay were performed by incubating 250 μl of pathogens (4.10⁸ CFU/ml) with 250 μl of Lactobacillus supernatant and 500 μl of DMEM 1 h at 37° C. Appropriate dilutions were plated on TSA to determine viable bacteria by bacteria colony count.

For inhibition of cell association and cell invasion, cells were infected with 1 ml of preincubating (1 h, 37° C.) pathogens with supernatant. After 1 h (Salmonella) or 3 h (DAEC) incubation at 37° C. 10% CO₂, cells were washed three times with PBS. In order to determine the cell-associated bacteria (extracellular+intracellular bacteria), the infected cell monolayers were lysed by adding H₂O. Appropriate dilutions were plated on TSA to determine the number of viable cell-associated bacteria by bacterial colony counts.

Internalization of enteroinvasive bacteria was determined by quantitative determination of bacteria located within the infected monolayers using the aminoglycoside antibiotic assay. After incubation, monolayers were washed twice with sterile PBS and, afterwards, incubated 60 min in a medium containing 100 μg/ml gentamicin. Bacteria that adhere to the Caco-2/TC7 brush border were rapidly killed, whereas those located within Caco-2 cells were not. The monolayer was washed with PBS and lysed with sterilized H₂O. Appropriate dilutions were plated on TSA to determine the number of viable cell-intracellular bacteria by bacterial colony counts.

Each assay was conducted in triplicate with three successive passages of Caco-2 cells.

Results are shown in FIGS. 2, 4, and 5.

I.6. Antagonistic Activity Against Infected Caco-2 Cells:

Activity of LB-f-SCS against intracellular S. typhimurium or DAEC was determined using the pre-infected Caco-2. Differentiated Caco-2 cells were infected by S. typhimurium SL1344 (1 ml, 5.10⁷ CFU/ml, 1 hour) or DAEC (1 ml, 5.10⁷ CFU/ml, 3 hours). For Salmonella infection, after two washings of the cells with PBS, the extracellular bacteria were killed by gentamicin (50 μg/ml, 1 hour at 37° C.) and the infected cells were washed with PBS to remove the killed bacteria. PBS, MRS (for Man, Rogosa, Sharp) or LB-f-SCS were added apically and the cells were incubated for 1 hour at 37° C. Two hours after the end of treatment, determination of the viable intracellular S. typhimurium or viable adherent DAEC was conducted as previously.

Results are shown in FIGS. 8 and 9.

I.7. Helicobacter pylori:

To test the inhibition of H. pylori viability, a 36 h old culture in BHI agar was suspended in PBS and centrifuged; bacteria were suspended in BHI broth. Colony count assay were performed by incubating 250 μl of pathogens (4.10⁸ CFU/ml) with 250 μl of Lactobacillus supernatant and 500 μl of DMEM 1 h at 37° C. Appropriate dilutions were plated on BHI to determine viable bacteria by bacteria colony count.

Results are shown in FIG. 6.

Urease activity was determined by a method based on the commercial rapid urease test (RUT; Jatrox-test; Röhm-Pharma GmbH, Weiterstadt, Germany) with a sensitivity of 10² bacteria. Briefly, 10 μl of H. pylori culture were added to 1 ml of the reaction solution (urea 0.1 g/ml [wt/v] containing 17 μg of phenol red/ml [wt/v] as a pH indicator). The development of urease activity was measured as a function of time by a spectrophotometric analysis at 550 nm.

Results are shown in FIG. 7.

II. Results

A. Bacteria and Supernatants:

II.A.1. Bacterial Adhesion:

FIG. 1 shows that the living LB-f strain display a dose-dependent adhesiveness onto cultured human intestinal cells. In addition, killed LB-f bacteria display the same capacity of adhesion, although a slight decrease in level of adhesion was observed as compared with living bacteria.

II.A.2. Inhibitory Activity Against Adhesion of Radiolabeled Enterovirulent Bacteria:

FIG. 2 shows that both culture of living and killed LB-f bacteria exerted an inhibitory activity against adhesion of diffusely adhering E. coli (DAEC) strain C1845 onto cultured human intestinal cells. This activity is dose-dependent. Moreover, the same level of inhibitory activity is observed for control in DMEM or MRS-HCL pH 4.5.

II.A.3. Inhibitory Activity Against Invasion of Salmonella enterica Serovar Typhimurium:

FIG. 3 shows that culture of living LB-f bacteria inhibits the cell-invasion by Salmonella serovar Typhimurium within cultured human intestinal cells. Killed LB-f bacteria exerted an inhibitory activity.

B. Supernatants:

II.B.1. Antibacterial Activity:

a) Against Salmonella enterica Serovar Typhimurium.

FIG. 4 shows that the spent culture supernatant (SCS) of living LB-f bacteria and the heated SCS decreased the viability of S. typhimurium strain. This activity is similar that activity of LB strain. The SCS of living LB-f bacteria and the heated SCS decreased the adhesion onto and internalization within cultured human intestinal cells by S. typhimurium strain. These activities are similar that activities of LB strain.

b) Against DAEC:

FIG. 5 shows that the SCS of living LB-f bacteria and the heated SCS slightly decreased the viability of diffusely adhering E. coli (DAEC) strain C1845 and strongly decreased adhesion of pathogenic E. coli onto cultured human intestinal cells.

c) Against Helicobacter pylori:

FIG. 6 shows that the SCS of living LB-f bacteria and the heated SCS strongly decreased the viability of Helicobater pylori.

FIG. 7 shows that the SCS of living LB-f bacteria and the heated SCS strongly decreased the urease activity of Helicobater pylori.

II.B.2. Cell-Association and Cell-Invasion of Enteroinvasive Bacteria:

a) Cell-Invasion of Salmonella enterica Serovar Typhimurium:

FIG. 8 shows that the SCS of living LB-f bacteria and the heated SCS strongly decreased the level of living, internalized S. typhimurium bacteria within primarily infected cultured human intestinal cells.

b) Cell-Association of DAEC C1845:

FIG. 9 shows that the SCS of living LB-f bacteria and the heated SCS strongly decreased the level of adhering, living diffusely adhering E. coli C1845 bacteria into primarily infected cultured human intestinal cells.

### REFERENCES

Finlay, B. B., and S. Falkow. 1997. Common themes in microbial pathogenicity revisited. Microbiol. Mol. Biol. Rev. 61:136-169.
Corthésy-Theulaz, I., N. Porta, M. Glauser, E. Saraga, A. C. Vaney, R. Haas, J. P. Kraehenbuhl, A. L. Blum, and P. Michetti. 1995. Oral immunisation with Helicobacter pylori urease B subunit as a treatment against Helicobacter infection in mice. Gastroenterology 109:115-121.
Fogh, J., J. M. Fogh, and T. Orfeo. 1977. One hundred and twenty seven cultured human tumor cell lines producing tumors in nude mice. J. Natl. Cancer Res. 59:221-226.
Chantret, I., M. Lacasa, G. Chevalier, D. Swallow, and M. Rousset. 1993. Monensin and forskolin inhibit the transcription rate of sucrase-isomaltase but not the stability of its mRNA in Caco-2 cells. FEBS Lett. 328:55-58.
Zweibaum, A., M. Laburthe, E. Grasset, and D. Louvard. Use of cultured cell lines in studies of intestinal cell differentiation and function. 1991. In Handbook of Physiology. The Gastrointestinal System, volume IV, Intestinal Absorption and Secretion, Schultz S J, Field M, Frizell R A, eds, American Physiological Society, Bethesda. 223-255.
Smith and Waterman. 1981. J. Theor. Biol. 91(2):370-380.
Needleman and Wunsh. 1972. J. Mol. Biol. 48(3):443-453.
Pearson and Lipman. 1988. PNAS USA. 85(5):2444-2448.

1. A Lactobacillus fermentum strain (LB-f strain), deposited at the CNCM (Paris, France) on Mar. 27, 2003, under registration number I-2998.

2. The LB-f strain according to claim 1, having at least the following phenotypic characters:
regular, non sporing, Gram-positive rod;
heterofermenting;
catalase negative; and
L (+)-lactic acid-producing.

3. The LB-f strain according to claim 1, having a 16S rDNA sequence comprising a nucleotide sequence selected from:
SEQ ID No. 1;
its complementary sequence ; and
sequences identical at least at 98.1% to SEQ ID No. 1 or to its complementary sequence.

4. The LB-f strain according to claim 3, wherein said sequences are identical at least at 98.5%, and preferably at least at 99% to SEQ ID No. 1 or to its complementary sequence.

5. The LB-f strain according to claim 4, wherein said sequences are identical at least at 99.5%, and preferably at least at 99.8% to SEQ ID No. 1 or to its complementary sequence.

6. A method for cultivating a Lactobacillus fermentum strain (LB-f strain) according to claim 1, comprising at least:
a) providing a culture medium containing at least lactose and yeast extract;
b) cultivating said LB-f strain in said culture medium under fermenting conditions; and
c) recovering the thus obtained culture of the LB-f strain.

7. The method according to claim 6, wherein said culture medium contains lactose at a concentration range of about 50 to about 100 g/l.

8. The method according to claim 6, wherein said culture medium contains yeast extract at a concentration range of about 5 to about 20 g/l.

9. The method according to claim 6, wherein said fermenting conditions in step b) are pH-regulated, said pH ranging between about 4.5 and 5.5.

10. The method according to claim 6, further comprising separating the biomass from the culture supernatant (LB-f-SCS) by centrifugating said culture of LB-f strain recovered in step c).

11. The method according to claim 10, further comprising recovering said biomass and/or said LB-f-SCS.

12. A Lactobacillus fermentum culture supernatant (LB-f-SCS) obtainable by a method according to claim 11.

13-23. (canceled)

24. A pharmaceutical composition comprising a Lactobacillus fermentum strain (LB-f strain) according to claim 1, and a pharmaceutically acceptable carrier.

25. The pharmaceutical composition according to claim 24, wherein said LB-f strain is present in an amount from about 10⁹ to about 10¹² bacteria/g, preferably from about 10⁹ to about 10¹¹ bacteria/g, and more preferably from about 10⁹ to about 10¹⁰ bacteria/g.

26. A pharmaceutical composition comprising a Lactobacillus fermentum culture supernatant (LB-f-SCS) according to claim 12, and a pharmaceutically acceptable carrier.

27. The pharmaceutical composition according to claim 26, wherein said LB-f-SCS is present in an amount of at least about 100 mg per gram of composition.

28. The pharmaceutical composition according to claim 24, wherein said composition is ingestible.

29. The pharmaceutical composition according to claim 28, wherein said composition is in a form selected from tablets, liquid bacterial suspensions, dried oral supplements, wet oral supplements, dry tube feeding, wet tube feeding.

30. A dietary composition comprising a Lactobacillus fermentum strain (LB-f strain) according to claim 1, and a food carrier.

31. The dietary composition according to claim 30, wherein said LB-f strain is present in an amount from about 10⁵ to about 10⁹ bacteria/g, preferably from about 10⁶ to about 10⁸ bacteria/g, and more preferably from about 10⁶ to about 10⁷ bacteria/g.

32. A dietary composition comprising a Lactobacillus fermentum culture supernatant (LB-f-SCS) according to claim 12, and a food carrier.

33. The dietary composition according to claim 32, wherein said LB-f-SCS is present in an amount of less than about 100 mg per gram of composition.

34. The dietary composition according to claim 30, wherein said dietary composition is ingestible.

35. The dietary composition according to claim 34, wherein said composition is selected from milk, yogurt, curd, cheese, fermented milks, fermented milk-based products, ice-creams, fermented cereal-based product, milk-based powders, infant formulae.

36. A method for treating or preventing gastrointestinal disorders in a mammal, especially a human, in need of such treatment, said method comprising:
administering to said mammal a pharmaceutically effective amount of a medicine selected from the group of:
a Lactobacillus fermentum strain (LB-f strain) according to claim 1;
a Lactobacillus fermentum culture supernatant (LB-f-SCS) according to claim 12; and
a pharmaceutical composition according to claim 24.

37. The method according to claim 36, wherein said gastrointestinal disorders are selected from ulcers and infections due to Helicobacter pylori, intestinal inflammatory diseases, such as ulcerous colitis, Crohn's disease and pouchitis, irritable bowel syndrome, steatohepatitis, hepatic steatosis, and infectious diarrhoea.

38. The method according to claim 36, wherein said medicine is administered orally.

39. The method according to claim 38, wherein said medicine is in a form selected from tablets, liquid bacterial suspensions, dried oral supplements, wet oral supplements, dry tube feeding, wet tube feeding.


SEQUENCE LISTING (1 sequence)

SEQ ID NO: 1 - DNA, 1500 bp, Lactobacillus fermentum
  16S rDNA
gaacgccggcggtgtgcctaatacatgcaagtcgaacgcgttggcccaattgattgatggtgcttgcacctgattgattttggtcgccaacgagtggcggacgggtgagtaacacgtaggtaacctgcccagaagcgggggacaacatttggaaacagatgctaataccgcataacaacgttgttcgcatgaacaacgcttaaaagatggcttctcgctatcacttctggatggacctgcggtgcattagcttgttggtggggtaatggcctaccaaggcgatgatgcatagccgagttgagagactgatcggccacaatgggactgagacacggcccatactcctacgggaggcagcagtagggaatcttccacaatgggcgcaagcctgatggagcaacaccgcgtgagtgaagaagggtttcggctcgtaaagctctgttgttaaagaagaacacgtatgagagtaactgttcatacgttgacggtatttaaccagaaagtcacggctaactacgtgccagcagccgcggtaatacgtaggtggcaagcgttatccggatttattgggcgtaaagagagtgcaggcggttttctaagtctgatgtgaaagccttcggcttaaccggagaagtgcatcggaaactggataacttgagtgcagaagagggtagtggaactccatgtgtagcggtggaatgcgtagatatatggaagaacaccagtggcgaaggcggctacctggtctgcaactgacgctgagactcgaaagcatgggtagcgaacaggattagataccctggtagtccatgccgtaaacgatgagtgctaggtgttggagggtttccgcccttcagtgccggagctaacgcattaagcactccgcctggggagtacgaccgcaaggttgaaactcaaaggaattgacgggggcccgcacaagcggtggagcatgtggtttaattcgaagctacgcgaagaaccttaccaggtcttgacatcttgcgccaaccctagagatagggcgtttccttcgggaacgcaatgacaggtggtgcatggtcgtcgtcagctcgtgtcgtgagatgttgggttaagtcccgcaacgagcgcaacccttgttactagttgccagcattaagttgggcactctagtgagactgccggtgacaaaccggaggaaggtggggacgacgtcagatcatcatgccccttatgacctgggctacacacgtgctacaatggacggtacaacgagtcgcgaactcgcgagggcaagcaaatctcttaaaaccgttctcagttcggactgcaggctgcaactcgcctgcacgaagtcggaatcgctagtaatcgcggatcagcatgccgcggtgaatacgttcccgggccttgtacacaccgcccgtcacaccatgagagtttgtaacacccaaagtcggtggggtaaccttttaggagccagccgcctaaggtgggacagatgattagggtgaagtc